(19) United States
(12) Patent Application Publication
(10) Pub. No.: US 2021/0104326 A1
(43) Pub. Date: Apr. 8, 2021

(54) DETECTING PRESCRIPTION DRUG ABUSE USING A DISTRIBUTED LEDGER AND MACHINE LEARNING

(71) Applicant: INTERNATIONAL BUSINESS MACHINES CORPORATION, ARMONK, NY (US)

(72) Inventors: Mario J. Lorenzo, Miami, FL (US); Thembani Togwe, Lenexa, KS (US); Komminist Weldemariam, Ottawa (CA); Manivannan Thavasi, Rochester, MN (US)

(21) Appl. No.: 16/592,818

(22) Filed: Oct. 4, 2019

(51) Int. Cl. G16H 50/30; G16H 10/60; G16H 20/10; G06N 20/00

(52) U.S. Cl. CPC G16H 50/30; G06N 20/00; G16H 20/10; G16H 10/60

(57) ABSTRACT

Methods, systems, and computer software product are provided to receive a request to encode a prescription for a patient on a blockchain ledger. A request is received to encode patient pick-up information for the prescription on the blockchain ledger. Based on the patient pick-up information, whether a request to fill a prescription is valid is evaluated. The blockchain ledger is scanned within a window of time for patient patterns of behavior indicating possible prescription drug abuse by the patient. Also provided is computing a score for the patient, the score representing a likelihood of fraud or abuse by the patient. A disposition for the request to fill the prescription is determined, based on a consensus of voting peers, and the disposition is recorded on the blockchain ledger.

### TECHNICAL FIELD

The present invention relates generally to the field of computing, and more particularly, to blockchain.

### BACKGROUND

Processes to prevent unauthorized access to controlled substance prescriptions are often circumvented, particularly with regard to the class of drugs referred to as opioids, or other co-actor drugs or enhancers. As a result, deaths resulting from this unauthorized access are increasing world-wide. However, currently a single aggregated view of controlled substance prescriptions, which may mitigate unauthorized access to controlled substance prescriptions, does not exist.

### SUMMARY

A method is provided to receive a request to encode a prescription for a patient on distributed ledger technologies such as blockchains. A request is received to encode patient pick-up information for the prescription on the blockchain ledger. Based on the patient pick-up information, whether a request to fill a prescription is valid is evaluated. The blockchain ledger is scanned within a window of time for patient patterns of behavior indicating possible prescription drug abuse by the patient. Also provided is computing a score for the patient, the score representing a likelihood of fraud or abuse by the patient. A disposition for the request to fill the prescription is determined, based on a consensus of voting peers, and the disposition is recorded on the blockchain ledger.

Another example embodiment provides a computer program product for detecting drug abuse, comprising a computer-readable storage medium having computer-readable program code embodied therewith, the computer-readable program code when executed on a computer causes the computer to receive a request to encode patient pick-up information for the prescription on the blockchain ledger. Based on the patient pick-up information, evaluate whether a request to fill a prescription is valid. The program code scans the blockchain ledger within a window of time for patient patterns of behavior indicating possible prescription drug abuse by the patient, and computes a score for the patient, wherein the score represents a likelihood of fraud or abuse by the patient. The program code determines a disposition for the request to fill the prescription, based on a consensus of voting peers, and records the disposition on the blockchain ledger.

Yet another example embodiment provides a simulation model based on a sequence neural net (such as a Long Short-Term Memory LSTM) that may learn the progression of how drug prescriptions or cohort drugs' prescriptions evolve based on training examples of how drug prescriptions evolve. Such a model may then take as input a reference drug prescription sequence and new presented drug prescriptions and determine whether the new presented prescriptions is a legitimate progression of the reference drug prescription. In one embodiment, the re-training itself may make use of blockchain analytics. For example, by entering drug prescription IDs or drug prescriptions into a blockchain for reverse correlation, specific verification steps (requiring accurate detection or prediction in order to verify or falsify a presented ID and generate appropriate action automatically) may then trigger access to and analysis of the blockchain retrospectively. This selective look back into blocks obviates the need for continuous tracking of all valid or invalid prescriptions over time for drugs, and instead maintains data and information privacy such that only those actions are accessed leading up to specific verification steps. The reverse correlation is a narrow description of a broader set of statistical machine techniques that can be applied to the look back. For example: Principal Component Analysis, Singular Value Decomposition, Deep Learning, LSTM based sequence learning, etc.

### BRIEF DESCRIPTION OF THE SEVERAL VIEWS OF THE DRAWINGS

FIG. 1 illustrates an example peer node blockchain architecture configuration, according to example embodiments.

FIG. 2 illustrates an example blockchain configuration, according to example embodiments.

FIG. 3 illustrates a flow diagram of encoding a prescription using blockchain according to example embodiments.

FIG. 4 illustrates a flow diagram of a clearinghouse performing consensus authentication of a request to fill a prescription.

FIG. 5 is a block diagram of internal and external components of computers and servers depicted in FIG. 1 according to at least one embodiment.

FIG. 6 is a block diagram of an illustrative cloud computing environment including the computer system depicted in FIG. 1, in accordance with an embodiment of the present disclosure.

FIG. 7 is a block diagram of functional layers of the illustrative cloud computing environment of FIG. 5, in accordance with an embodiment of the present disclosure.

### DETAILED DESCRIPTION

It will be readily understood that the instant components, as generally described and illustrated in the figures herein, may be arranged and designed in a wide variety of different configurations. Thus, the following detailed description of the embodiments of one or more method, apparatus, non-transitory computer readable medium and system, as represented in the attached figures, is not intended to limit the scope of the application as claimed but is merely representative of selected embodiments.

The instant features, structures, or characteristics as described throughout this specification may be combined in any suitable manner in one or more embodiments. For example, the usage of the phrases “example embodiments”, “some embodiments”, or other similar language, throughout this specification refers to the fact that a particular feature, structure, or characteristic described in connection with the embodiment may be included in one or more embodiments. Thus, appearances of the phrases “example embodiments”, “in some embodiments”, “in other embodiments”, or other similar language, throughout this specification do not necessarily all refer to the same group of embodiments, and the described features, structures, or characteristics may be combined in any suitable manner in one or more embodiments.

Example embodiments provide methods, devices, networks and/or systems, which provide detecting unauthorized access to prescriptions for controlled substances, i.e., drugs. While national and state agencies require strict compliance with increasingly strict regulations, unauthorized access to controlled substance prescriptions, particularly to the opioid class of drugs, is increasing. There are other co-actor drugs or enhancers such as Gabapentin which are not opioids but when taken with opioids increase the euphoric effects. These “enhancers” are increasingly added to the controlled substances state surveillance programs. It typically takes months or years of research, scientific debate, and tragic outcomes before they are eventually added to the Federal controlled substance list or to standard drug tests.

Deaths resulting from this unauthorized access are also increasing. Currently, various governmental agencies store in databases various information relating to controlled substances, such as prescribers, the patients, and the class of drugs. However, a single aggregated view of the usage of controlled substances as a single point of truth generated from decentralized networks of computing peers and databases, which may mitigate unauthorized access to controlled substance prescriptions, does not exist. This results in states and agencies maintaining different databases, with information sharing being voluntary among them. This makes it possible for a patient to evade detection by existing drug monitoring programs by crossing state lines to seek a prescription by another doctor. In the United States, federal agencies may attempt to aggregate this data, but the collection process tends to focus on retrospective statistics reporting and lacks predictive and detection capabilities for proactively intervening to stop abuse. The discontinuity between referring providers, and pharmacies can mask multiple redundant prescriptions within or across state lines.

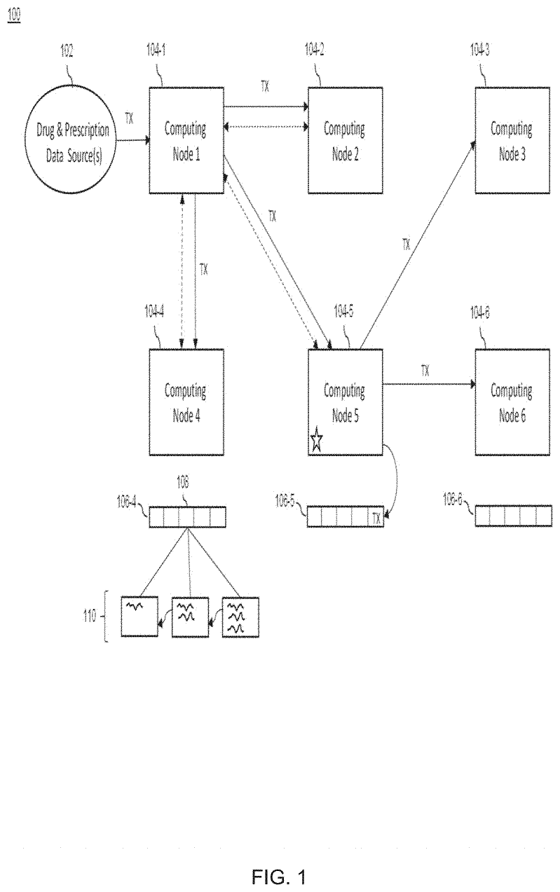

FIG. 1 illustrates a blockchain computational system 100 with which one or more embodiments of the invention may be implemented. As shown, the system 100 comprises one or more data sources 102 operatively coupled to at least one of a plurality of distributed peer computing nodes 104-1, 104-2, . . . , 104-6. The system 100 may have more or less computing nodes than the number illustrated in FIG. 1. Each computing node in the system 100 is configured to maintain a blockchain which is a cryptographically secured (via a cryptographic hash function) record or ledger of data blocks that represent transactions within some environment. A cryptographic hash function is a cryptographic function which takes an input (or “message”) and returns a fixed-size alphanumeric string, which is called the hash value (sometimes called a message digest, a digital fingerprint, a digest, or a checksum).

In FIG. 1, computing nodes 104-4, 104-5, and 104-6 are shown each maintaining the same blockchain (respectively illustrated as blockchains 106-4, 106-5, and 106-6). Although not expressly shown, each computing node in the system 100 is configured to be able to maintain this same blockchain. Each blockchain is a growing list of data records hardened against tampering and revision (i.e., secure). Each block in the blockchain (illustratively referenced as block 108 in blockchain 106-4) holds batches of one or more individual transactions and the results of any blockchain executables (e.g., computations that can be applied to the transactions). Each block typically contains a timestamp and information linking it to a previous block. More particularly, each subsequent block in the blockchain (e.g., 106-4, 106-5, 106-6, etc.) is a data block that includes a given transaction and a hash value of the previous block in the chain (i.e., the previous transaction). The current transaction and the hash value of the prior transactions can itself be hashed to generate a hash value. Thus, each data block in the blockchain represents a given set of transaction data plus a set of all previous transaction data (e.g., as illustratively depicted as 110 in FIG. 1).

Assume a new set of transaction data (new transaction TX) is obtained from one of the one or more data sources 102, and received by computing node 1 (104-1). Computing node 1 (104-1) can provide the new transaction TX to all or a subset of computing nodes in the system 100. In this case, TX is sent to computing node 2 (104-2), computing node 4 (104-4), and computing node 5 (104-5).

Note that computing node 104-5 is marked with a star symbol to denote it as a leader in a consensus protocol. That is, the computing nodes in the system 100 each are configured to participate in a consensus protocol as peers with one peer being designated as a leader. Any peer can assume the role of leader for a given iteration of the consensus protocol. In general, the leader receives all transactions from the participating peers in the system and creates a new block for the new transaction. The new block is sent out by the leader node to one or more of the other peer computing nodes (e.g., 104-3 and 104-6 as illustrated in FIG. 1) which double check (validate) that the leader computed the new block properly (i.e., the validating nodes agree by consensus). If consensus is reached, then the computing nodes in the system 100 add the new block to the blockchain they currently maintain. As a result, after the new transaction TX is processed by the system 100, each computing node should now have a copy of the same updated blockchain stored in its memory. Then, when another new transaction comes into the system 100, the above-described process of adding the transaction to the blockchain is repeated.

It is to be understood that any single computing node may itself serve as the receiver, validator, and block generator of a new transaction data set. However, in the context of a consensus protocol, the more nodes that validate the given transaction, the more trustworthy the data block is considered.

It is to be further understood that the above description represents one illustrative blockchain computation process and that embodiments of the invention are not limited to the above or any particular blockchain computation protocol and/or implementation. As such, other appropriate cryptographic processes may be used to maintain and add to a secure ledger of data in accordance with embodiments of the invention.

Advantages of such a blockchain computational system include, but are not limited to: (1) the ability for independent nodes to converge on a consensus of a latest version of a large data set (e.g., a ledger), even when the nodes are run anonymously, have poor interconnectivity and may have operators who are dishonest or otherwise malicious; (2) the ability for any well-connected node to determine, with reasonable certainty, whether a transaction does or does not exist in the data set; (3) the ability for any node that creates a transaction to, after a confirmation period, determine with a reasonable level of certainty whether the transaction is valid, able to take place, and become final (i.e., that no conflicting transactions were confirmed into the blockchain elsewhere that would invalidate the transaction); (4) a prohibitively high cost to attempt to rewrite or otherwise alter transaction history; and (5) automated conflict resolution that ensures that conflicting transactions never become part of the confirmed data set.

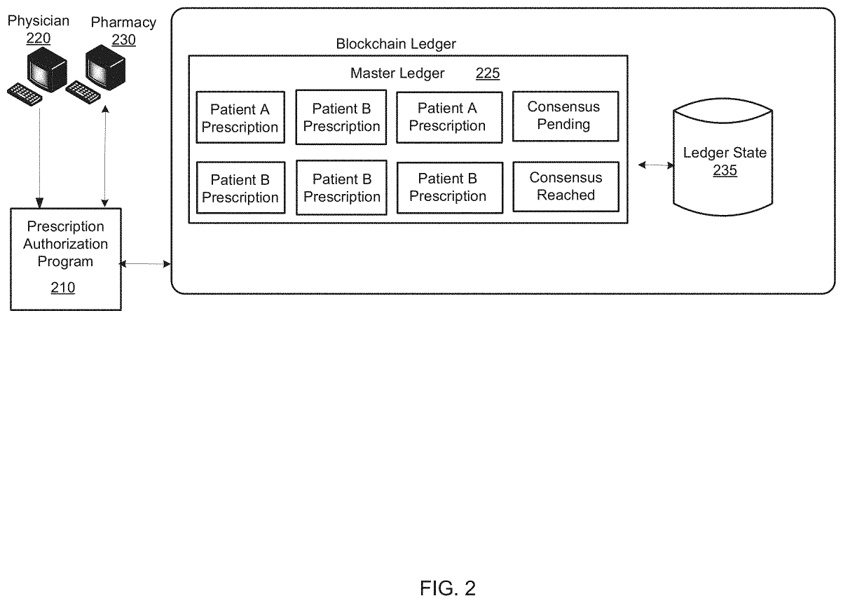

FIG. 2 illustrates an example blockchain configuration, according to example embodiments. Referring to FIG. 2, the blockchain configuration includes a prescription authorization program 210. A plurality of users, two of which are shown as physician 220 and pharmacy 230 are connected to the prescription authorization program 210. The prescription authorization program 210 monitors transactions from the users, and based on configured definitions, determines whether the transactions, particularly the pharmacy 230 transaction, may be executed. Through the prescription authorization program 210, the physician 220 initiates a transaction that encodes a prescription for a patient on the blockchain ledger 225. In the interest of patient privacy and security, it is not necessary for the physician 220 to evaluate the authenticity of the patient's request. The US Department of Health recommends, as part of good clinical practice, that prescribers or dispensers check/query the prescription drug monitoring program (e.g., the State database) every time a controlled substance is prescribed or dispensed. However, not all states have mandatory enrollment for both physicians/prescribers and pharmacists/dispensers. Only historic information is available.

Since sharing of prescription drug use data among government agencies is currently limited, it may not be practical to attempt. However, using a generated model to predict the patient's likelihood of fraud or abuse, the prescription authorization program 210, communicating with the clearinghouse (i.e., a peer node) determines the validity of the prescription. For example, four transactions for Patient B were encoded on the blockchain ledger 225. The consensus process, using two or more prescription ledger clearinghouses (clearinghouse) arrived at a consensus, which may be to reject the patient's ability to fill the prescription at any pharmacy. However, Patient A may be permitted to fill the prescription request, depending on the results of the pending consensus process and Patient A risk score. Blockchain transactions are immutable, but may be traceable through entries in the ledger state 235. The method of traceable through entities in the ledger state is by scanning the blockchain ledger within a window of time for the patient patterns of behavior that will indicate possible prescription drug abuse by prescribers, the patients, and the class of drugs.

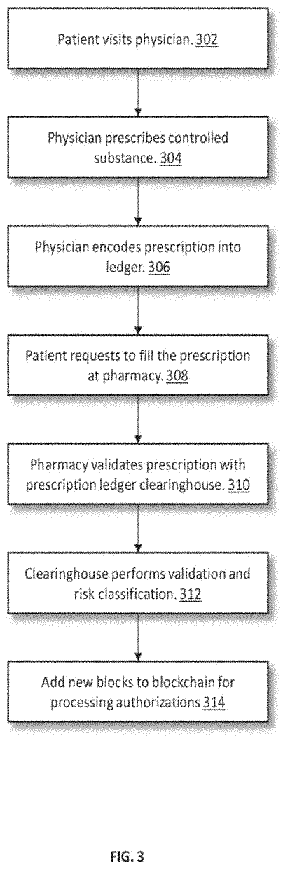

FIG. 3 illustrates and example flow diagram of encoding a prescription using blockchain according to example embodiments.

Starting at 302, a patient visits a physician and receives a diagnosis. The circumstance of the patient's visit may be innocuous. For example, the patient may be a new resident in the area and is beginning a relationship with this physician. Similarly, the physician may be beginning a new practice. However, in a practice known as doctor shopping, the patient may have targeted this physician in an attempt to illicitly obtain a prescription for a controlled drug (i.e., controlled substance).

At 304, the physician evaluates the patient's symptoms and as a result, arrives at a diagnosis that a prescription for a controlled substance is warranted.

At 306, the physician encodes the prescription into the blockchain ledger 225, using the prescription authorization program 210. In one embodiment, the encoded prescription includes the physician's Drug Enforcement Agency (DEA) number, the controlled substance name and dosage, and patient de-identified information, including patient ID, transformed name, address, age, and unique identifying number. The DEA number is an identifier that is assigned to a health care provider, including a physician ID, optometrist, dentist, or veterinarian, by the United States Drug Enforcement Administration. The DEA number authorizes the health care provider to write prescriptions for controlled substances. Some of the encoded information may be implementation dependent. It should be noted that the prescription authorization program 210 and the clearinghouses can process physician identifying numbers from other international agencies by providing their formats and links. For example, some jurisdictions may recognize a taxpayer identification number, while some may recognize another type of national token. A hash value can be derived from some or all of the encoded information. The identifying patient hash can comprise some or all of the patient information, while the identifying physician information can comprise some or all of the physician information. The hash value provides additional security for patient personal information. This is because a comparison of the encoded information on the blockchain ledger 225 to that encoded at the pharmacy will fail if either sets of encoded information is altered. The prescription information is encrypted and is appended, along with the hash value, to the blockchain ledger 225. A reference and hash of scanned image of the prescription is appended to the blockchain ledger.

At 308, the patient requests that the prescription is filled at any pharmacy. Because the patient prescription information is stored as a transaction on the blockchain ledger 225, as opposed to being maintained at different levels of information sharing across state and federal agencies, the patient can choose any pharmacy. However, the patient's attempts to seek anonymity at a pharmacy where he is not known (i.e., pharmacy shopping) is defeated precisely because of the nature of the blockchain ledger 225.

At 310, the pharmacy uses the prescription authorization program 210 to encode a prescription pick-up order request on the blockchain ledger 225. The pick-up order request is used to validate the patient's prescription against the prescription information originally encoded on the blockchain ledger 225. The pick-up order request includes the same information that originally created the prescription request, coupled with current timestamp. The purpose of this request is to cross-validate and authenticate the information entered with the encoded prescription by scanning and reverse correlating blocks using one or more machine learning algorithms, thereby enhancing data security.

At 312, the prescription authorization program 210 notifies the clearinghouse that a pick-up order request was encoded on the blockchain ledger 225, and that consensus processing is required on the validity of the prescription before the pharmacy can proceed. Two or more clearinghouses are required to validate request to fill a prescription using a consensus decision for authorization and risk analysis against the patient historical records, for example, analyzing side-effects on controlled substance based on drug-patient interactions, drug-drug interactions and so on. As an example of harmful drug-drug interactions, non-opioid drugs are increasingly being added to state and federal controlled substances list because they have been determined to enhance the euphoric effects of the opioid. The consensus process is globally distributed and decentralized, allowing for rapid processing time. The consensus process can be implemented as any consensus type algorithm, such as lottery-based and voting based. The risk analysis can be preconfigured to each peer node of the blockchain network and can be managed using smart contract. The number and choice of clearinghouses required to reply to the request for consent for consensus is a pre-configured rule in the smart contract but at least two clearinghouses are required. The purpose of the pre-configured rule is to prevent sequence of attempts to circumvent anonymity and impartiality in the consensus process. For example, either all, or only a subset of the clearinghouses, are required to approve filling the prescription. As another example, the request for consent for consensus can be sent to the closest clearinghouse to the entity initiating the request, the next in a sequential order, or randomly. In one embodiment, new robust rules can be learned from historical rules and past performance of the historical rules.

At 314, the clearinghouse consensus decision is relayed to the entity that initiated the consensus request, usually the pharmacy. The consensus decision, and the transaction to fill the prescription, along with timestamps, are added to the blockchain ledger 225.

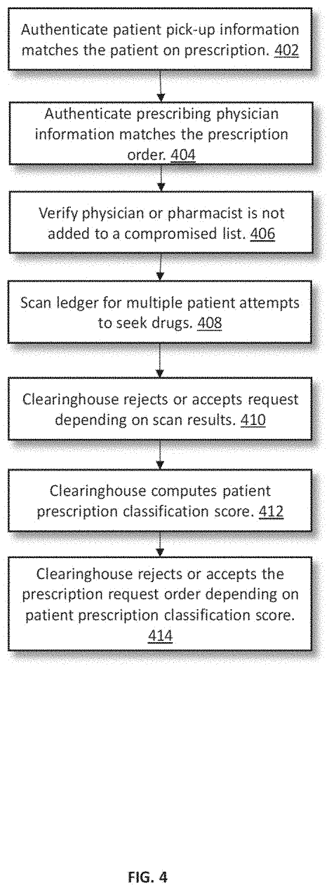

FIG. 4 illustrates a flow diagram of a clearinghouse performing consensus authentication of a request to fill a prescription. The clearinghouse process is a cross authentication of the prescription information encoded by the physician. Each clearinghouse that participates in the consensus performs this authentication process.

At 402, the clearinghouse authenticates the patient pick-up information against the patient information that the physician encoded when creating the prescription. The clearinghouse creates a hash value of some or all of the patient pick-up information, including one or more forms of patient identities. The clearinghouse uses the same combination of patient information the prescription authorization program 210 used to encode the prescription on the blockchain ledger 225. The clearinghouse compares the two hash values. A mismatch is a factor that the clearinghouse can use in its consensus vote.

At 404, the clearinghouse authenticates the information of the physician that encoded the prescription. The clearinghouse creates a hash value of some or all of the physician information. The clearinghouse uses the same combination of physician information that the prescription authorization program 210 used to encode the prescription on the blockchain ledger 225. The clearinghouse compares the two hash values. A mismatch is another factor that the clearinghouse can use in its consensus vote. The clearinghouse performs a more focused verification of the DEA number. This is because detecting or probability of a forged prescription at a pharmacy is relatively easy, given the rules comprising the DEA number, and can provide an initial defense against fraud. Anomaly detection machine learning models can be trained and deployed at each peer node to detect forged prescription. To verify a DEA number, the clearinghouse performs a series of calculations or runs the anomaly detection models to ensure that the DEA number that is encoded in the prescription more likely than not belongs to the physician named in the prescription. For example, using a check digit in the calculation is one way to detect a forged prescription. Similarly, the DEA number includes information that represents the physician's unique code and type of practice. A mismatch in the DEA number indicates a fraud that the clearinghouse reports back to the prescription authorization program 210 as a failure vote.

At 406, the clearinghouse focuses on the physician DEA number to verify whether the physician is on a list of compromised physicians. This can be done automatically by machine learning model pre-configured with a peer node. The focus on the DEA number can provide an initial defense against fraud and is relatively easy, given the rules comprising the DEA number. To verify a DEA number, the clearinghouse performs a series of calculations to ensure that the DEA number that is encoded in the prescription more likely than not belongs to the physician named in the prescription. For example, using a check digit in the calculation is one way to detect a forged prescription. Similarly, the DEA number includes information that represents the physician's unique identifier and type of practice. Additionally, the clearinghouse can access various repositories of compromised healthcare providers, such as the Centers for Medicare and Medicaid Services (CMS). In this context, a compromised health care provider is one whose identities are stolen or are being used fraudulently. A mismatch in the DEA number to physician, or the inclusion of the DEA number on a list of compromised physicians, indicates a fraud that the clearinghouse reports back to the prescription authorization program 210 as a failure vote.

At 408, the clearinghouse scans the blockchain ledger 225 for other activity by the patient attempting the pick-up. For example, the clearinghouse may search a sliding six month, or another implementation defined, window. The clearinghouse is searching for patterns of behavior, such as sequences of multiple attempts to obtain the drug that are beyond normal use patterns, or sequence of attempts to obtain the drug from multiple sources, such as urgent care and hospital emergency services. In one embodiment, a sequence mining machine learning model is trained.

At 410, the clearinghouse can vote to reject or accept the pick-up request, based on the scan results. For example, in FIG. 2 after a scan of the blockchain ledger 225 for Patient B's activity consensus was reached. The result was likely to reject the prescription pick-up because of the patient's multiple attempts that were outside the normal use patterns for the drug.

At 412, the clearinghouse calculates a patient prescription calculation score (score) to represent the likelihood of fraud or abuse by this patient. A machine learning model generates the score for each patient prescription. For example:

ŷ_{fraudScore}(w,x)=w₀+w₁x_{stm_prec_context}+w₂x_{stm_demographic}+w₃x_{stm_age}+w₄x_{gender}+w₅x_{income}+ . . .

ŷ_{pre_fraud_score}(w,x)

Where:

ŷ_{pre_fraud_score}(w,x)

is the estimated prescription score for a patient with respect to representing the likelihood of fraud or abuse by this patient. The drug prescription context may consist of recent activity, class of drug being prescribed, dose, etc.

The vector of coefficients is represented by:

w=(w0,w1,w3,w4,w5, . . . )

The vector of inputs is represented by:

x=(x_{stm_prec_context},x_{stm_demographic},x_{stm_age},x_{gender},x_{income}, . . . )

The computed score is added to the blockchain ledger 225. The score is computed using known statistical methods such as a logistic regression model or deep neural network model that is trained to compare this patient's activity with similar peer or cohort group population activity. In this way, the model can identify whether the patient is an outlier or is behaving within statistical norms. Inputs to the model include the patient information, such as that encoded on the prescription, demographic information, such as age, gender, and income. The model also considers the drug and class of drug, for example opioid, and dose.

The clearinghouse uses known statistical methods, such as a multinomial classification machine learned model to place this patient within a risk category (high, medium, low). For example, if the patient is in the statistically normal distribution, the patient is in a medium risk category. If above or below a standard deviation, then the patient is in a high or low risk category, respectively. The model leverages features that represent patient information, recent activity, class of drug being prescribed, and dose. It should be noted that these are not the only methods and algorithms for training on a patient's activity. Other considerations include: the required degree of accuracy in the result; the amount of time available for training; the number of parameters in the algorithm (i.e., error tolerance, number of iterations); and the degree of linearity in the data (i.e., whether the classes of data can be represented by a straight line).

This model is actively retrained using data gathered to identify normal activity from potential abuse and fraud activity. This model is actively retrained using data gathered to identify normal activity from potential abuse and fraud activity. Anomalous pattern detection is the task of identifying subsets of data points that systematically differ from the underlying model. For example, early identification of anomalous patterns in a patient's prescription drug usage can potentially lead to an intervention which can prevent developing a pattern of abuse.

The model can be retrained using Gaussian process and subset scanning. Subset scanning is a framework for detecting events and other patterns in both spatial and nonspatial data, through constrained optimization of a score function (e.g., a likelihood ratio statistic) over subsets of the data. By combining information across a subset of data elements, subset scanning methods indicate anomalous behavior. Subset scanning methods compute a log-likelihood ratio (LLR) that can be used to measure how much evidence exists in the data to conclude abnormal behavior. Gaussian processes provide a non-parametric approach to regression problems by finding a distribution over the possible functions that are consistent with the observed data.

In another embodiment, the pharmacy can provide feedback to the clearinghouse that can additionally train the model. For example, a patient may have a prescription from both a dentist and a physician for the same drug that treats the same condition. This can result in a rejection which is encoded in the blockchain ledger 225, but also trigger feedback to the pharmacy to verify the prescription with the physician/dentist. In this case, the duplicate prescriptions may be for two different forms of the drug, and therefore are necessary. The model is updated to know that this patient may appear to be in a high risk category for a period of time, but there are mitigating factors. As described previously, the model can be trained for mitigating factors using Gaussian processes and subset scanning.

The model is continuously being updated. When the model is initially deployed and activity begins to flow into the model there is not enough data to determine a statistically significant pattern. Therefore, initially every transaction will look like an outlier. While it is possible to use existing data, such as clinical trial result from the United States Federal Drug Administration, or similar international agencies, this is not as effective as allowing the model to self-train. This is because the existing data is a retrospective measurement and may not accurately reflect the actual population. The model is trained initially using a pre-encoded heuristic/rule which can be defined in collaboration with domain experts, using existing procedures, processes and/or guidelines. These heuristic/rules will be translated to smart contracts. Over time when enough historical data is collected, the transition from rule based to model based operation can be made. The domain experts can assist in validating the models. During training mode after a pre-defined threshold is reached, such as 95% accuracy, the model can begin operating in active mode. Therefore, during the training period existing drug monitoring programs should be relied upon to flag possible prescription drug abuse situations. Similarly, a new class of drug entering the market triggers re-training of the model because initially there will not be enough patient usage to be statistically significant. In this case, all orders can be accepted and the existing drug monitoring programs should be relied upon to flag possible prescription drug abuse situations.

At 414, the clearinghouse accepts or rejects the prescription pick-up request order, depending on the patient score. If the consensus from the voting clearinghouses results in a failure vote, then the transaction to pick-up the prescription is rejected. The prescription authorization program 210 can initiate a transaction to the national or international drug enforcement agency to report possible fraud. The various cross-border rules can be encoded in the smart contracts.

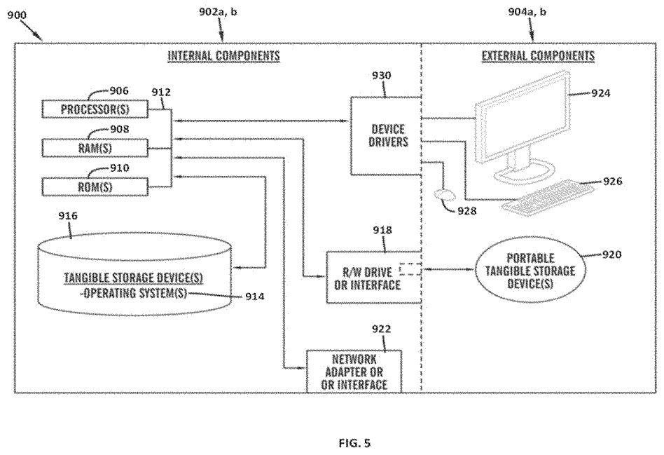

FIG. 5 is a block diagram 900 of internal and external components of computers depicted in FIG. 1 in accordance with an illustrative embodiment of the present invention. It should be appreciated that FIG. 5 provides only an illustration of one implementation and does not imply any limitations with regard to the environments in which different embodiments may be implemented. Many modifications to the depicted environments may be made based on design and implementation requirements.

Data processing system 902, 904 is representative of any electronic device capable of executing machine-readable program instructions. Data processing system 902, 904 may be representative of a smart phone, a computer system, PDA, or other electronic devices. Examples of computing systems, environments, and/or configurations that may represented by data processing system 902, 904 include, but are not limited to, personal computer systems, server computer systems, thin clients, thick clients, hand-held or laptop devices, multiprocessor systems, microprocessor-based systems, network PCs, minicomputer systems, and distributed cloud computing environments that include any of the above systems or devices.

Node computers 102 may include respective sets of internal components 902 a, b and external components 904 a, b illustrated in FIG. 5. Each of the sets of internal components 902 a, b includes one or more processors 906, one or more computer-readable RAMs 908 and one or more computer-readable ROMs 910 on one or more buses 912, and one or more operating systems 914 and one or more computer-readable tangible storage devices 916. The one or more operating systems 914 and the prescription authorization program 210 may be stored on one or more computer-readable tangible storage devices 916 for execution by one or more processors 906 via one or more RAMs 908 (which typically include cache memory). In the embodiment illustrated in FIG. 5, each of the computer-readable tangible storage devices 916 is a magnetic disk storage device of an internal hard drive. Alternatively, each of the computer-readable tangible storage devices 916 is a semiconductor storage device such as ROM 910, EPROM, flash memory or any other computer-readable tangible storage device that can store a computer program and digital information.

Each set of internal components 902 a, b also includes a R/W drive or interface 918 to read from and write to one or more portable computer-readable tangible storage devices 920 such as a CD-ROM, DVD, memory stick, magnetic tape, magnetic disk, optical disk or semiconductor storage device. A software program, such as the prescription authorization program 210 can be stored on one or more of the respective portable computer-readable tangible storage devices 920, read via the respective R/W drive or interface 918 and loaded into the respective hard drive 916.

Each set of internal components 902 a, b may also include network adapters (or switch port cards) or interfaces 922 such as a TCP/IP adapter cards, wireless wi-fi interface cards, or 3G or 4G wireless interface cards or other wired or wireless communication links. The prescription authorization program 210 in node computer 102 can be downloaded from an external computer (e.g., server) via a network (for example, the Internet, a local area network or other, wide area network) and respective network adapters or interfaces 922. From the network adapters (or switch port adaptors) or interfaces 922, the prescription authorization program 210 are loaded into the respective hard drive 916. The network may comprise copper wires, optical fibers, wireless transmission, routers, firewalls, switches, gateway computers and/or edge servers.

Each of the sets of external components 904 a, b can include a computer display monitor 924, a keyboard 926, and a computer mouse 928. External components 904 a, b can also include touch screens, virtual keyboards, touch pads, pointing devices, and other human interface devices. Each of the sets of internal components 902 a, b also includes device drivers 930 to interface to computer display monitor 924, keyboard 926 and computer mouse 928. The device drivers 930, R/W drive or interface 918 and network adapter or interface 922 comprise hardware and software (stored in storage device 916 and/or ROM 910).

It is understood in advance that although this disclosure includes a detailed description on cloud computing, implementation of the teachings recited herein are not limited to a cloud computing environment. Rather, embodiments of the present invention are capable of being implemented in conjunction with any other type of computing environment now known or later developed.

Cloud computing is a model of service delivery for enabling convenient, on-demand network access to a shared pool of configurable computing resources (e.g. networks, network bandwidth, servers, processing, memory, storage, applications, virtual machines, and services) that can be rapidly provisioned and released with minimal management effort or interaction with a provider of the service. This cloud model may include at least five characteristics, at least three service models, and at least four deployment models.

Characteristics are as follows:

On-demand self-service: a cloud consumer can unilaterally provision computing capabilities, such as server time and network storage, as needed automatically without requiring human interaction with the service's provider.

Broad network access: capabilities are available over a network and accessed through standard mechanisms that promote use by heterogeneous thin or thick client platforms (e.g., mobile phones, laptops, and PDAs).

Resource pooling: the provider's computing resources are pooled to serve multiple consumers using a multi-tenant model, with different physical and virtual resources dynamically assigned and reassigned according to demand. There is a sense of location independence in that the consumer generally has no control or knowledge over the exact location of the provided resources but may be able to specify location at a higher level of abstraction (e.g., country, state, or datacenter).

Rapid elasticity: capabilities can be rapidly and elastically provisioned, in some cases automatically, to quickly scale out and rapidly released to quickly scale in. To the consumer, the capabilities available for provisioning often appear to be unlimited and can be purchased in any quantity at any time.

Measured service: cloud systems automatically control and optimize resource use by leveraging a metering capability at some level of abstraction appropriate to the type of service (e.g., storage, processing, bandwidth, and active user accounts). Resource usage can be monitored, controlled, and reported providing transparency for both the provider and consumer of the utilized service.

Service Models are as follows:

Software as a Service (SaaS): the capability provided to the consumer is to use the provider's applications running on a cloud infrastructure. The applications are accessible from various client devices through a thin client interface such as a web browser (e.g., web-based e-mail). The consumer does not manage or control the underlying cloud infrastructure including network, servers, operating systems, storage, or even individual application capabilities, with the possible exception of limited user-specific application configuration settings.

Platform as a Service (PaaS): the capability provided to the consumer is to deploy onto the cloud infrastructure consumer-created or acquired applications created using programming languages and tools supported by the provider. The consumer does not manage or control the underlying cloud infrastructure including networks, servers, operating systems, or storage, but has control over the deployed applications and possibly application hosting environment configurations.

Analytics as a Service (AaaS): the capability provided to the consumer is to use web-based or cloud-based networks (i.e., infrastructure) to access an analytics platform. Analytics platforms may include access to analytics software resources or may include access to relevant databases, corpora, servers, operating systems or storage. The consumer does not manage or control the underlying web-based or cloud-based infrastructure including databases, corpora, servers, operating systems or storage, but has control over the deployed applications and possibly application hosting environment configurations.

Infrastructure as a Service (IaaS): the capability provided to the consumer is to provision processing, storage, networks, and other fundamental computing resources where the consumer is able to deploy and run arbitrary software, which can include operating systems and applications. The consumer does not manage or control the underlying cloud infrastructure but has control over operating systems, storage, deployed applications, and possibly limited control of select networking components (e.g., host firewalls).

Deployment Models are as follows:

Private cloud: the cloud infrastructure is operated solely for an organization. It may be managed by the organization or a third party and may exist on-premises or off-premises.

Community cloud: the cloud infrastructure is shared by several organizations and supports a specific community that has shared concerns (e.g., mission, security requirements, policy, and compliance considerations). It may be managed by the organizations or a third party and may exist on-premises or off-premises.

Public cloud: the cloud infrastructure is made available to the general public or a large industry group and is owned by an organization selling cloud services.

Hybrid cloud: the cloud infrastructure is a composition of two or more clouds (private, community, or public) that remain unique entities but are bound together by standardized or proprietary technology that enables data and application portability (e.g., cloud bursting for load-balancing between clouds).

A cloud computing environment is service oriented with a focus on statelessness, low coupling, modularity, and semantic interoperability. At the heart of cloud computing is an infrastructure comprising a network of interconnected nodes.

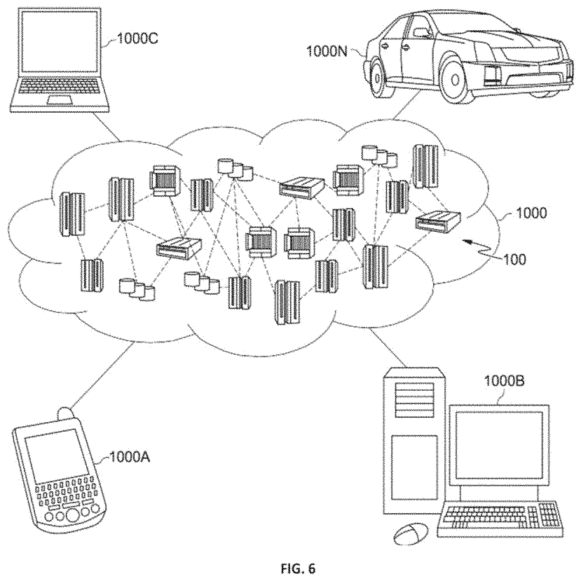

Referring now to FIG. 6, illustrative cloud computing environment 1000 is depicted. As shown, cloud computing environment 1000 comprises one or more cloud computing nodes 100 with which local computing devices used by cloud consumers, such as, for example, personal digital assistant (PDA) or cellular telephone 1000A, desktop computer 1000B, laptop computer 1000C, and/or automobile computer system 1000N may communicate. Nodes 100 may communicate with one another. They may be grouped (not shown) physically or virtually, in one or more networks, such as Private, Community, Public, or Hybrid clouds as described hereinabove, or a combination thereof. This allows cloud computing environment 1000 to offer infrastructure, platforms and/or software as services for which a cloud consumer does not need to maintain resources on a local computing device. It is understood that the types of computing devices 1000A-N shown in FIG. 4 are intended to be illustrative only and that computing nodes 100 and cloud computing environment 1000 can communicate with any type of computerized device over any type of network and/or network addressable connection (e.g., using a web browser).

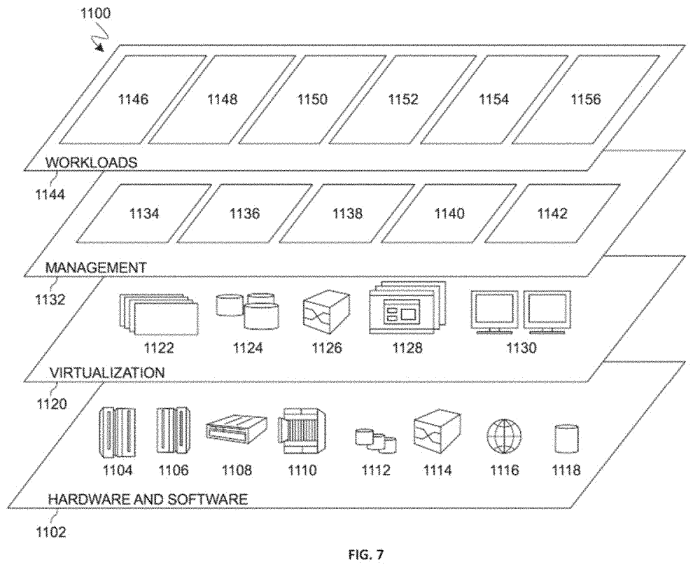

Referring now to FIG. 7, a set of functional abstraction layers 1100 provided by cloud computing environment 1000 is shown. It should be understood in advance that the components, layers, and functions shown in FIG. 7 are intended to be illustrative only and embodiments of the invention are not limited thereto. As depicted, the following layers and corresponding functions are provided:

Hardware and software layer 1102 includes hardware and software components. Examples of hardware components include: mainframes 1104; RISC (Reduced Instruction Set Computer) architecture based servers 1106; servers 1108; blade servers 1110; storage devices 1112; and networks and networking components 1114. In some embodiments, software components include network application server software 1116 and database software 1118.

Virtualization layer 1120 provides an abstraction layer from which the following examples of virtual entities may be provided: virtual servers 1122; virtual storage 1124; virtual networks 1126, including virtual private networks; virtual applications and operating systems 1128; and virtual clients 1130.

In one example, management layer 1132 may provide the functions described below. Resource provisioning 1134 provides dynamic procurement of computing resources and other resources that are utilized to perform tasks within the cloud computing environment. Metering and Pricing 1136 provide cost tracking as resources are utilized within the cloud computing environment, and billing or invoicing for consumption of these resources. In one example, these resources may comprise application software licenses. Security provides identity verification for cloud consumers and tasks, as well as protection for data and other resources. User portal 1138 provides access to the cloud computing environment for consumers and system administrators. Service level management 1140 provides cloud computing resource allocation and management such that required service levels are met. Service Level Agreement (SLA) planning and fulfillment 1142 provide pre-arrangement for, and procurement of, cloud computing resources for which a future requirement is anticipated in accordance with an SLA.

Workloads layer 1144 provides examples of functionality for which the cloud computing environment may be utilized. Examples of workloads and functions which may be provided from this layer include: mapping and navigation 1146; software development and lifecycle management 1148; virtual classroom education delivery 1150; data analytics processing 1152; transaction processing 1154; and pre-authorization 1156. A pre-authorization program 110a, 110b provides a way to reduce patient wait times by using NLP and ML combined with key features of successful treatments to identify key stakeholders for authorization.

The present invention may be a system, a method, and/or a computer program product at any possible technical detail level of integration. The computer program product may include a computer readable storage medium (or media) having computer readable program instructions thereon for causing a processor to carry out aspects of the present invention.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present invention may be assembler instructions, instruction-set-architecture (ISA) instructions, machine instructions, machine dependent instructions, microcode, firmware instructions, state-setting data, configuration data for integrated circuitry, or either source code or object code written in any combination of one or more programming languages, including an object oriented programming language such as Smalltalk, C++, or the like, and procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instruction by utilizing state information of the computer readable program instructions to personalize the electronic circuitry, in order to perform aspects of the present invention.

Aspects of the present invention are described herein with reference to flowchart illustrations and/or block diagrams of methods, apparatus (systems), and computer program products according to embodiments of the invention. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments of the present invention. In this regard, each block in the flowchart or block diagrams may represent a module, segment, or portion of instructions, which comprises one or more executable instructions for implementing the specified logical function(s). In some alternative implementations, the functions noted in the blocks may occur out of the order noted in the Figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by software or hardware-based systems that perform the specified functions or acts or carry out combinations of computer instructions.

The descriptions of the various embodiments of the present invention have been presented for purposes of illustration, but are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments described herein.

What is claimed is:

1. A computer-implemented method for detecting drug abuse, comprising:
receiving a request to encode a prescription for a patient on a blockchain ledger;
receiving a request to encode patient pick-up information for the prescription on the blockchain ledger;
based on the patient pick-up information, evaluating whether a request to fill a prescription is valid;
scanning the blockchain ledger within a window of time for patient patterns of behavior indicating possible prescription drug abuse by the patient;
computing a score for the patient, wherein the score represents a likelihood of fraud or abuse by the patient; and
determining a disposition for the request to fill the prescription, based on a consensus of voting peers, and recording the score and the disposition on the blockchain ledger.

2. The method of claim 1, wherein the encoded prescription information includes: a unique physician identifier, a controlled substance name, a class of drug, a dosage, secured reference of patient identifying information, and a hash value that is derived from some or all of the encoded prescription information, and wherein the hash value is stored with the encoded prescription on the blockchain ledger.

3. The method of claim 1, wherein evaluating further comprises:
comparing a hash value that is derived from some or all of the encoded prescription information to a hash value that is derived from some or all of the patient pick-up information, wherein a mismatch is a factor in the consensus of voting peers; and
verifying that the unique physician identifier is not on a list of compromised identifiers.

4. The method of claim 1, wherein detecting the patterns of behavior include establishing sequence of attempts to obtain a prescription drug that are outside normal patterns of use, establishing sequence of attempts to obtain the prescription drug from multiple sources, wherein multiple sources include urgent care, and hospital emergency services.

5. The method of claim 1, wherein computing the score for the patient further comprises:
training a machine learning model, using patient information, wherein the patient information includes recent patient activity, class of drug, and dosage;
comparing the patient patterns of behavior with activity of a peer-group, wherein the comparison indicates whether the patient is behaving within statistical norms; and
based on the score, assigning the patient a risk category.

6. The method of claim 5, wherein the patient is in a normal, low, or high risk category, based on the patient being in a statistically normal distribution, a standard deviation below normal distribution, or a standard deviation above normal distribution, respectively.

7. The method of claim 1, wherein in response to a consensus vote to reject the prescription or to reject the request to fill the prescription, a transaction is generated to report the rejection to a drug enforcement agency.

8. A system for detecting drug abuse, comprising:
one or more processors;
a memory coupled to at least one of the processors;
computer program instructions stored in the memory and executed by at least one of the processors in order to cause the processor to:
receive a request to encode a prescription for a patient on a blockchain ledger;
receive a request to encode patient pick-up information for the prescription on the blockchain ledger;
based on the patient pick-up information, evaluate whether a request to fill a prescription is valid;
scan the blockchain ledger within a window of time for patient patterns of behavior indicating possible prescription drug abuse by the patient;
compute a score for the patient, wherein the score represents a likelihood of fraud or abuse by the patient; and
determine a disposition for the request to fill the prescription, based on a consensus of voting peers, and recording the score and the disposition on the blockchain ledger.

9. The system of claim 8, wherein the encoded prescription information includes: a unique physician identifier, a controlled substance name, a class of drug, a dosage, secured reference of patient identifying information, and a hash value that is derived from some or all of the encoded prescription information, and wherein the hash value is stored with the encoded prescription on the blockchain ledger.

10. The system of claim 8, wherein the evaluate further comprises:
compare a hash value that is derived from some or all of the encoded prescription information to a hash value that is derived from some or all of the patient pick-up information, wherein a mismatch is a factor in the consensus of voting peers; and
verify that the unique physician identifier is not on a list of compromised identifiers.

11. The system of claim 8, wherein the patterns of behavior include attempts to obtain a prescription drug that are outside normal patterns of use, attempts to obtain the prescription drug from multiple sources, wherein multiple sources include urgent care, and hospital emergency services.

12. The system of claim 8, wherein the compute of the score for the patient further comprises:
train a machine learning model, using patient information, wherein the patient information includes recent patient activity, class of drug, and dosage;
compare the patient patterns of behavior with activity of a peer-group, wherein the comparison indicates whether the patient is behaving within statistical norms; and
based on the score, assigning the patient a risk category.

13. The system of claim 12, wherein the patient is in a normal, low, or high risk category, based on the patient being in a statistically normal distribution, a standard deviation below normal distribution, or a standard deviation above normal distribution, respectively.

14. The system of claim 8, wherein in response to a consensus vote to reject the prescription or to reject the request to fill the prescription, a transaction is generated to report the rejection to a drug enforcement agency.

15. A computer program product for detecting drug abuse, comprising a computer-readable storage medium having computer-readable program code embodied therewith, the computer-readable program code when executed on a computer causes the computer to:
receive a request to encode patient pick-up information for the prescription on the blockchain ledger;
based on the patient pick-up information, evaluate whether a request to fill a prescription is valid;
scan the blockchain ledger within a window of time for patient patterns of behavior indicating possible prescription drug abuse by the patient;
compute a score for the patient, wherein the score represents a likelihood of fraud or abuse by the patient; and
determine a disposition for the request to fill the prescription, based on a consensus of voting peers, and recording the score and the disposition on the blockchain ledger.

16. The computer program product of claim 15, wherein the encoded prescription information includes: a unique physician identifier, a controlled substance name, a class of drug, a dosage, secured reference of patient identifying information, and a hash value that is derived from some or all of the encoded prescription information, and wherein the hash value is stored with the encoded prescription on the blockchain ledger.

17. The computer program product of claim 15, wherein the evaluate further comprises:
compare a hash value that is derived from some or all of the encoded prescription information to a hash value that is derived from some or all of the patient pick-up information, wherein a mismatch is a factor in the consensus of voting peers; and
verify that the unique physician identifier is not on a list of compromised identifiers.

18. The computer program product of claim 15, wherein the compute of the score for the patient further comprises:
train a machine learning model, using patient information, wherein the patient information includes recent patient activity, class of drug, and dosage;
compare the patient patterns of behavior with activity of a peer-group, wherein the comparison indicates whether the patient is behaving within statistical norms; and
based on the score, assigning the patient a risk category.

19. The computer program product of claim 18, wherein the patient is in a normal, low, or high risk category, based on the patient being in a statistically normal distribution, a standard deviation below normal distribution, or a standard deviation above normal distribution, respectively.

20. The computer program product of claim 15, wherein in response to a consensus vote to reject the prescription or to reject the request to fill the prescription, a transaction is generated to report the rejection to a drug enforcement agency.
